(19) United States
(12) Patent Application Publication
(10) Pub. No.: US 2010/0249530 A1
(43) Pub. Date: Sep. 30, 2010

(54) Bolus Estimator with Image Capture Device

(75) Inventors: Ulrich Rankers, Livermore, CA (US); Andrew Michael Bryan, Los Angeles, CA (US); Eric S. Chen, Santa Monica, CA (US)

Correspondence Address: MEDTRONIC MINIMED INC., 18000 DEVONSHIRE STREET, NORTHRIDGE, CA, 91325-1219, US

(73) Assignee: MEDTRONIC MINIMED, INC., Northridge, CA (US)

(21) Appl. No.: 12/606,398

(22) Filed: Oct. 27, 2009

Related U.S. Application Data
(60) Provisional application No. 61/162,819, filed on Mar. 24, 2009.

(51) Int. Cl. A61B 5/00

(52) U.S. Cl. 600/300

(57) ABSTRACT

A method of providing bolus dosage recommendations for diabetics includes receiving an image of a meal to be consumed by a user. The image is analyzed to identify at least one food item in the image. A bolus dosage recommendation is calculated based on the identified at least one food item in the image.

### FIELD OF THE INVENTION

Embodiments of the present invention are directed to systems and methods for estimating an insulin bolus. Specifically, embodiments of the present invention are directed to a bolus estimator/calculator having an image capture device to analyze an image of a meal to be consumed and calculate a bolus dosage recommendation.

### BACKGROUND OF THE INVENTION

Insulin must be provided to people with Type I and many with Type II diabetes. Traditionally, since it cannot be taken orally, insulin has been injected with a syringe. More recently, use of external infusion pump therapy has been increasing, especially for delivering insulin for diabetics using devices worn on a belt, in a pocket, or the like, with the insulin delivered via a catheter with a percutaneous needle or cannula placed in the subcutaneous tissue. For example, as of 1995, less than 5% of Type I diabetics in the United States were using pump therapy. There are about 10% of the currently over 1.5 million Type I diabetics in the U.S. using insulin pump therapy, and the percentage is now growing at an absolute rate of over 2% each year. Moreover, the number of Type I diabetics is growing at 3% or more per year. In addition, growing numbers of insulin using Type II diabetics are also using external insulin infusion pumps. Physicians have recognized that continuous infusion provides greater control of a diabetic's condition and are also increasingly prescribing it for patients. In addition, medication pump therapy is becoming more important for the treatment and control of other medical conditions, such as pulmonary hypertension, HIV, and cancer. Although offering control, pump therapy can suffer from several complications that make use of a pump less desirable for the user.

A drawback for diabetic pump users, in particular, is the determination of the amount of bolus insulin to be delivered for a meal so as to avoid high blood sugars that would otherwise be caused by the meal. This determination can be a difficult calculation using formulas and approximations that have several variables that must be measured and calculated. Often, it is easier, but not the best for control, for the user to simply guess what they need rather than to calculate the actual amount of the bolus needed to adequately cover the carbohydrates being consumed. However, in worse case scenarios, guessing can lead to under or overdosing of medication, sometimes with dire consequences.

### SUMMARY OF THE INVENTION

A method of providing bolus dosage recommendations for diabetics includes receiving an image of a meal to be consumed by a user. The image is analyzed to identify at least one food item in the image. A bolus dosage recommendation is calculated based on the identified at least one food item in the image.

The image may be captured by a digital camera. Moreover, a portion size of the at least one food item in the image may be determined, and the portion size of the at least one food item in the image may be determined by analyzing a reference object of a known size in the image. A carbohydrate value of the identified at least one food item in the image may be determined and obtained from a database. The image of the meal to be consumed may be compared with food images stored in a database to identify the at least one food item. The food images stored in the database may include corresponding known carbohydrate values. At least one of a carbohydrate value, a protein value, a fat value, and a caloric value of the identified at least one food item in the image may be determined. A current blood glucose level of the user may be received. Embodiments of the present invention may be implemented on a medical system.

A bolus estimator includes an image capture device to capture an image of a meal to be consumed by a user. A processor is operatively coupled to the image capture device to analyze the image to identify at least one food item in the image, and to calculate a bolus dosage recommendation based on the identified at least one food item in the image. A display is operatively coupled to the processor to display the bolus dosage recommendation.

The processor may determine a portion size of the at least one food item in the image. The portion size of the at least one food item in the image may be determined by analyzing a reference object of a known size in the image. The processor may determine a carbohydrate value of the identified at least one food item in the image. The carbohydrate value may be obtained from a database. The processor may access a database to compare the image with food images stored in the database to identify the at least one food item. The food images stored in the database may include corresponding known carbohydrate values. The processor may determine at least one of a carbohydrate value, a protein value, a fat value, and a caloric value of the identified at least one food item in the image. The bolus estimator may be integrated into a controller, an infusion pump, or a blood glucose meter. The bolus estimator may be integrated into a mobile phone, a personal digital assistant (PDA), or a portable computer. The bolus estimator may include an input device to receive a current blood glucose level of the user. The image capture device may be a digital camera.

### BRIEF DESCRIPTION OF THE DRAWINGS

FIG. 1 illustrates a block diagram of a bolus estimator according to embodiments of the present invention.

FIG. 2 illustrates a flow chart operation of a bolus estimator according to embodiments of the present invention.

### DETAILED DESCRIPTION

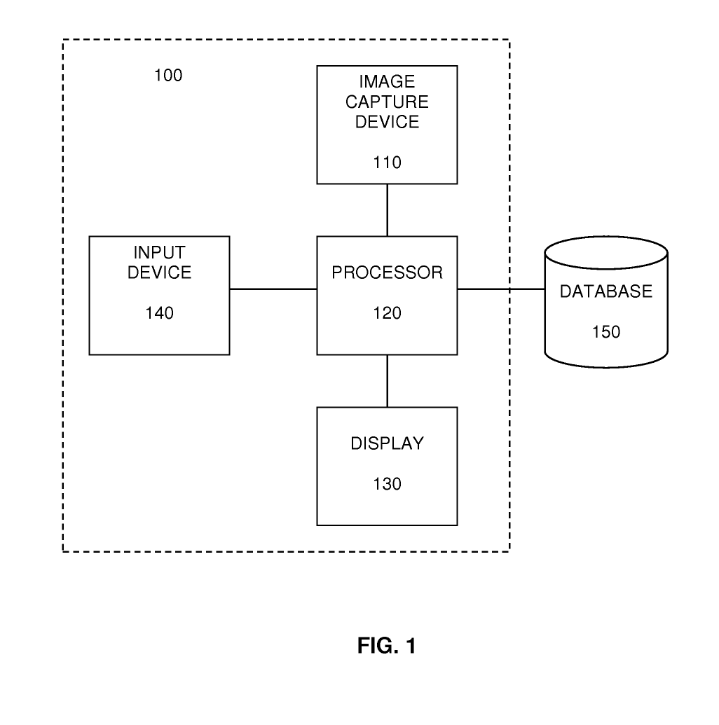

FIG. 1 illustrates a block diagram of a bolus estimator according to embodiments of the present invention. A bolus estimator/calculator (or carbohydrate estimator/calculator that estimates an insulin bolus based on carbohydrate consumption (CHO)) assists a user with carbohydrate counting, and/or meal portion calculation, and in determining precise insulin dosing adjustments to account for meals. Carbohydrates are the primary, but not the only, factor affecting blood glucose levels. According to embodiments of the present invention, values other than carbohydrate values of a meal/beverage to be consumed, e.g., protein values, fat values, caloric values, etc., may be utilized by a bolus estimator/calculator to calculate bolus dosage recommendations. Generally, it is sufficient to account just for the carbohydrates. It also encourages the user to enter current blood glucose values before using this feature, which also will be viewed quite favorably by the health care professional, since it increases compliance with the medical regimen and improves control. In alternative embodiments, the bolus estimator 100 may be connected or coupled to a glucose monitor (not illustrated) by way of a programmer (or other data transfer) to provide direct input to the bolus estimator 100.

According to embodiments of the present invention, the bolus estimator 100 may be used to assist an external infusion device (not illustrated) user with the estimations that are done to determine the proper bolus amount that is needed to cover the anticipated carbohydrate intake at meals. The bolus estimator 100 does this by suggesting a bolus based on a pre-programmed carbohydrate ratio that is stored in the memory of the bolus estimator 100, or the like. The bolus estimator 100 also may take into account the user's insulin sensitivity and the differential between the user's pre-programmed target blood glucose (BG) level and the user's current BG level at the time the bolus estimator 100 is activated. The recommendation, or result of the bolus estimator 100, is sometimes referred to as a “correction bolus”.

The bolus estimator 100 may be integrated with an external infusion device, or the like, and is generally activated by the user, or preferably the health care professional, in a set-up menu of the external infusion device, before it is operational, and preferably after the user has demonstrated a sufficient understanding of estimating carbohydrate intake. According to various embodiments of the present invention, the bolus estimator 100 may be activated and programmed by using an input device 140, such as a keypad. In alternative embodiments, the bolus estimator 100 may be programmed and activated with another device such as a programmer/controller, or the like. In further alternative embodiments, the current glucose readings for the user may be provided by receipt of the glucose level measurement from a glucose monitor or via a programmer/controller to facilitate a correction for changing blood glucose (BG) levels. Further description of correcting infusion rates based on blood glucose readings may be found in U.S. Pat. No. 5,569,186 to Lord et al., entitled “Closed Loop Infusion Pump System with Removable Glucose Sensor”, and U.S. Pat. No. 5,665,065 to Colman et al., entitled “Medication Infusion Device with Blood Glucose Data Input”, which are herein incorporated by reference in their entirety. In alternative embodiments, the user may be able to use other combinations of the values to suggest different bolus types and amounts. In alternative embodiments, the bolus estimator 100 may be used in a closed-loop system to augment the readings or check the closed-loop system's capability based on carbohydrate estimated meals. In still further embodiments, the bolus estimator 100 may be used to calculate correction boluses based on other parameters, with the type of bolus corrections being determined by the fluid being infused, body characteristics, or the like. Preferably, the bolus estimator 100 uses stored values or parameters related to the individual with current values, parameters, or measurements and an algorithm to provide a recommended bolus that may be accepted, modified or rejected by the user. For instance in pregnancy, tocolysis may be infused and the measurement of the contraction rate may be used to suggest additional boluses of tocolysis medication. In HIV cases, a bolus amount of medication being infused may be adjusted based on a relationship to the current viral loads in the patient. In stroke or cardiac cases, the coagulation rate may be used to determine the bolus amount of heparin to be administered. Other calculations may be made and should not be limited to the above-described examples.

After the bolus estimator 100 has been enabled, the user may be prompted to store the following three values in the memory of the bolus estimator 100. In alternative embodiments, more or fewer values may be needed or used. These values are used by the processor 120 to perform the necessary calculations in suggesting a bolus amount. In preferred embodiments, access to programming and changing these values may be restricted to the health care professional. In alternative embodiments, these values may be restricted to entry through, for example, a programmer/controller or a connection of the bolus estimator 100 with a programming device, such as a PC, laptop or the like. The inputted values that may be stored for the bolus estimator 100 include:

Target Blood Glucose (Target), which is the target blood glucose (BG) that the user would like to achieve and maintain. Generally, the programmable blood glucose (BG) values for this range are between 60 to 200 in five unit increments. Preferably, the bolus estimator 100 has the capability to accept values that range between 20 to 600 in one-unit increments to cover a large number of possible scenarios. However, in alternative embodiments, different ranges and increments may be used.

Insulin Sensitivity (Set Sens), which is a value that reflects how far the user's blood glucose drops in milligrams per deciliter (mg/dl) when one unit of insulin is taken. Preferably, the programmable values for this range are between 5 to 180 in one-unit increments. However, in alternative embodiments, different ranges and increments may be used. In preferred embodiments, insulin sensitivity is programmable for up to four different time periods, the use of which will require four separate profiles to be stored in the memory. Setting the Insulin Sensitivity profiles is similar to setting the basal profiles. In alternative embodiments, more or fewer time periods (and corresponding profiles) may be used.

Carbohydrate Ratio (Set Carbs), which is a value that reflects the amount of carbohydrates that are covered by one unit of insulin. Generally, the values are in the range of 1 to 300 in increments of 1 unit (or, alternatively, in ranges of 0.1 to 5.0 in increments of 0.1 for carbohydrate exchanges). Preferably, the programmable values for this range are between 5 to 30 in one-unit increments. However, in alternative embodiments, different ranges and increments may be used.

As a safety precaution, the user or healthcare professional may also set a Lockout Period, which takes into account the pharmacokinetic effect of insulin when suggesting a bolus. The purpose is to prevent a successive use of a correction bolus when the pharmacokinetic effects of the previous bolus have not yet been accounted for. The programmable values for this range are between 30 minutes to 240 minutes, programmable in 15 or 30 minute increments. However, in alternative embodiments, different ranges and increments may be used. In further alternative embodiments, the lock out period may be automatically calculated based on boluses recently delivered and/or canceled based on new blood glucose (BG) readings. In other embodiments, the bolus estimator 100 may include a programmable reminder to check the post-prandial blood glucose value to determine if additional boluses and or corrections should be made at a later time after the meal. The programmable reminder values are between 30 minutes to 240 minutes, programmable in 15 or 30 minute increments. However, in alternative embodiments, different values and increments may be used.

After the above values are set in the memory, the bolus estimator 100 may suggest a bolus based on the entry of the estimated carbohydrate intake and current and target blood glucose (BG) levels. The calculation may be performed once the three values are programmed and stored in the memory. Embodiments of the present invention use the following equation:

Bolus=((CurrentBG−TargetBG)/Insulin Sensitivity)+(CarbohydratesToBeConsumed/CarbohydrateRatio))

If the user wishes the bolus estimator 100 to suggest a bolus for the estimated carbohydrate intake only, then the only value he/she needs to program is for the Carbohydrate Ratio, and the BG portion of the equation will be ignored. In alternative embodiments, variations or different equations may be used.

In operation, once the bolus estimator 100 has been enabled and the above listed values have been programmed into the memory, the bolus estimator 100 may be used to suggest a correction or meal bolus. The user may then accept or change the bolus amount suggested by the bolus estimator 100. According to embodiments of the present invention, the processor 120 stores in memory a record of whether the suggested bolus amount from the bolus estimator 100 was accepted or changed by the user, and records the suggested and changed bolus amounts. The stored data may be used for later analysis by downloading the data to a computer by wired or wireless transmissions.

According to embodiments of the present invention, the user sets a normal bolus for delivery. In alternative embodiments, the user may be given the choice of a normal, dual, square wave bolus, extended bolus, profiled bolus, or the like, by enabling these capabilities on a variable bolus menu on the bolus estimator 100 and/or external infusion device. If the variable bolus capability is not enabled, then every bolus would be a normal bolus. As discussed, embodiments of the present invention may use normal one time boluses. However, alternative embodiments may utilize different bolus types to spread out the correction or meal bolus determined by the carbohydrate estimator 100. Further description of bolus estimators/calculators may be found in U.S. Pat. No. 6,554,798, issued Apr. 29, 2003, to Mann et al., entitled, “External Infusion Device with Remote Programming, Bolus Estimator and/or Vibration Alarm Capabilities”, which is herein incorporated by reference in its entirety.

Referring to FIG. 1 and according to embodiments of the present invention, the bolus estimator 100 includes an image capture device 110, such as a digital/video camera, to capture an image and/or video of a meal and/or beverage to be consumed by a user. The image capture device 110 may be integrated into the housing of the bolus estimator 100. According to alternative embodiments of the present invention, the image capture device 110 (e.g., a camera) may be separate from the bolus estimator 100, that is, the bolus estimator 100 may be in one housing (and may receive an image or video of a meal/beverage from the image capture device 110 or from any other suitable device capturing/storing the image/video), and the image capture device 110 may be in another housing. For example, the image capture device 110 may be separate from, or integrated with, a blood glucose meter, an infusion device controller/programmer, an infusion pump, etc. A processor 120 is operatively coupled to the image capture device 110 to analyze the image/video and identify at least one food item (which may include beverages) in the image/video, and to calculate a bolus dosage recommendation based on the identified at least one food item in the image/video. Any suitable image recognition/image processing software (e.g., those used in facial recognition at public venues, airport security, casinos, and weapons target identification/acquisition, etc.) may be utilized to analyze the image of the meal to be consumed to identify at least one food item in the image. Once the at least one food item in the image has been identified, the bolus estimator 100 may access a database 150 (to be discussed in greater detail below) to determine the total carbohydrate value of the meal to be consumed, and a bolus dosage recommendation may be calculated based on the total carbohydrate value of the at least one food item in the image of the meal. According to alternative embodiments of the present invention, the image capture device 110 captures a video clip of the meal to be consumed, and the video clip may be further analyzed to identify at least one food item in the video clip. A video clip may be able to provide greater detail regarding the meal to be consumed than in a single non-moving image.

A display screen 130 is operatively coupled to the processor 120 to display the bolus dosage recommendation. The bolus estimator 100 may also include an input device 140 to receive a current blood glucose level of the user. The input device 140 may be a blood glucose meter to determine the user's current blood glucose level, and/or a keypad for the user to interface with the bolus estimator 100 to manually input the user's current blood glucose level, and/or a connection for wired/wireless coupling with another device, such as a blood glucose meter, to receive blood glucose level data. A transmitter, receiver, and/or transceiver (not shown) may be included with the bolus estimator 100 to facilitate communication with other devices, e.g., blood glucose meters, insulin infusion devices, PCs, mobile phones, portable computers, the Internet, etc.

According to embodiments of the present invention, the processor analyzes the image and determines a portion size of the at least one food item in the image. The processor may analyze the image for a reference object of a known size so as to determine the size of the at least one food item in the image, and calculate a portion size of the at least one food item based on the known size of the reference object in the image. For example, the reference object having a known size may be a coin, such as a quarter (or any suitable object), in the image/video, and the size and/or volume of the at least one food item in the image relative to the quarter may be analyzed to determine the actual portion size of the at least one food item. Knowing the portion size of the at least one food item permits greater accuracy in determining its carbohydrate value, which improves accuracy in calculating the bolus dosage recommendation. Any other suitable objects, such as napkins, utensils, cups, plates, jewelry, pens and pencils, etc., may be utilized as reference objects.

According to embodiments of the present invention, the processor 120 determines a carbohydrate value of the identified at least one food item in the image, and calculates the bolus recommendation based on the carbohydrate value of the identified at least one food item. Additional information, such as the user's insulin sensitivity, the user's current blood glucose level, the target blood glucose level, the carbohydrate ratio, etc., may be utilized for the calculation of the bolus recommendation. The bolus recommendation also may be calculated from any suitable information available about the meal to be consumed, such as sugar, carbohydrate type, protein value, fat value, caloric value, etc., and need not be solely based on carbohydrate values alone. The carbohydrate value may be obtained from a database 150, stored locally with the bolus estimator 100, and/or separately/remotely accessed (as illustrated in FIG. 1), for example, via wired or wireless connection (e.g., cellular network, WiFi, etc.), from the Internet, on a server, or any other suitable network or connection. Moreover, according to embodiments of the present invention, the processor 120 accesses a database 150, which may be the same or different from the one storing the carbohydrate values, locally and/or remotely, to compare the image/video with food images/videos stored in the database 150 to identify the at least one food item in the image. The food images stored in the database 150 may include information regarding known carbohydrate values of food items depicted in the food images.

Using any suitable image processing/recognition software, the image of the meal to be consumed is analyzed by the processor 120 to identify each food item in the image. The analysis may occur entirely locally at the bolus estimator 100, separately/remotely at another system (not illustrated), or occurring in a combination of both. According to embodiments of the present invention, the captured image of the meal to be consumed may be compared to existing images in a database 150 (locally and/or remotely) to identify the food item(s) in the captured image of the meal to be consumed. Once the food item(s) in the captured image of the meal to be consumed is identified (and their associated portion size(s), if applicable), corresponding carbohydrate values associated with the existing images in the database 150 may be retrieved to calculate a total carbohydrate count for the meal to be consumed; and based on this total carbohydrate count, a bolus dosage recommendation may be calculated. According to embodiments of the present invention, a “food library” of images with known carbohydrate values may be established in a database 150, and a user may download, for example, images from a “Top 50” food items and/or meals list of foods that the user routinely eats and/or enjoys onto the bolus estimator 100 for local storage in a local database 150 within the bolus estimator 100, and access a separate/remote database 150 for those food items not locally stored.

The bolus estimator 100 may be capable of identifying “set meals” (e.g., combination meals pre-configured by a restaurant) from a received image of a meal to be consumed. For example, the user captures with a digital camera an image of a meal to be consumed, and this image is received by the bolus estimator 100. By analyzing the image, the bolus estimator 100 determines that the meal is a JACK IN THE BOX® restaurant “Two Tacos Combo Meal” consisting of two regular beef tacos, a small order of seasoned curly fries, and a 20-oz. COCA COLA® soft drink. JACK IN THE BOX® is a trademark of Jack In The Box Inc., and COCA-COLA® is a trademark of The Coca-Cola Company. By identifying the set meal, the bolus estimator 100 may access a database 150 to obtain the total carbohydrate count of the food items in this set meal (e.g., 91 grams) without having to specifically identify each food item, obtain their corresponding carbohydrate counts, and obtain their total. Alternatively, the bolus estimator 100 may still identify each food item in an image of a set meal to confirm the carbohydrate values of each food item in the set meal, especially in situations where the user is likely to substitute, e.g., eight-piece onion rings (51 grams) in place of the curly fries (30 grams), and/or augment the portions, e.g., “supersize”—upgrade to the largest portions available for each food item in the set meal, of the food items, or subtle substitutions in portion size of one or more food items in the meal, e.g., a medium order of curly fries (45 grams) in place of the original small order of curly fries (30 grams) that comes with the set meal.

Accordingly to embodiments of the present invention, users of the bolus estimator 100 may join an Internet social network (e.g., MySpace.com, Facebook.com, LinkedIn.com, Friendster.com, etc.) to share their experiences and trade tips on improving their diabetes therapy. In this social network, users may trade images and/or video clips of food items and meals to be consumed (or upload them onto a forum and/or “photo library” for others to review and download, etc.), their portion size information, along with their corresponding carbohydrate counts, with other users. By sharing these images and/or video clips, users in the diabetic community may easily share carbohydrate information regarding food items and meals with each other, promoting greater and more accurate treatment opportunities, and users will not have to “reinvent the wheel” for food items and meals to be consumed where their carbohydrate information has already been determined.

Users may also rate (e.g., one to five stars) each available image as to how accurate and effective the carbohydrate information associated with that image was with respect to their therapy, and those images that are popularly rated would indicate that their associated carbohydrate information is most effective with users in their therapies, and these images with the best information will likely be better propagated to other users. Using this rating system, the images with the best carbohydrate information will tend to remain more relevant and popular, while those images with carbohydrate information that are not effective will be de-prioritized. For example, if a user had captured an image of the JACK IN THE BOX® restaurant “Two Tacos Combo Meal” (two regular beef tacos, a small order of seasoned curly fries, and a 20-oz. COCA COLA® soft drink), determined that the total carbohydrate count for this meal was 91 grams, and concluded that this carbohydrate count was accurate for the purposes of insulin therapy, then this image may be entitled, “Jack in the Box Two Tacos Combo”, the 91 grams total carbohydrate information may be associated therewith (e.g., embedded as metadata, imprinted onto the image, etc.), and eventually given a rating of “Five Stars” by the users if it is determined that the carbohydrate count was accurate for the purposes of insulin therapy for most of the users. Therefore, if other users know that they enjoy the “Two Tacos Combo Meal”, these users may simply download this existing image along with its carbohydrate information onto the database 150, and the analysis of the image of a “Two Tacos Combo Meal” by the bolus estimator 100 may be simplified. These “homemade” images of food items/meals may be shared and traded amongst users in the social network, or, these images may be professionally created, e.g., by the restaurants themselves, health care professionals, etc., to include the carbohydrate information, to further aid in insulin therapy.

Moreover, according to embodiments of the present invention, a database 150 may reside at a restaurant where the user is dining, and the bolus estimator 100 may have access to the database 150 at the restaurant of the food items available for purchase at that restaurant via wireless connection (e.g., radio frequency, Bluetooth, WiFi, RFID, cellular network, etc.), barcode scanner, or via any suitable connection.

According to embodiments of the present invention, the bolus estimator 100 may be integrated into an insulin infusion device/pump, a remote programmer/controller for the infusion device/pump, a blood glucose meter, or the like. Moreover, the bolus estimator 100 according to embodiments of the present invention may be integrated into a mobile/cellular phone (including those having a built-in digital camera), a personal digital assistant (PDA), a portable computer/laptop computer, or the like.

Users of the bolus estimator 100 according to embodiments of the present invention are provided with an expert system that aids the user in obtaining accurate bolus recommendations for a meal to be consumed, especially if the meal to be consumed has a difficult to determine carbohydrate count (e.g., the meal has various different ingredients), the food is not readily identifiable by the user, the user is not adept or skillful at counting/estimating carbohydrates in foods, or the like. Obtaining accurate bolus recommendations allows for better diabetes therapy in providing more stable glucose levels within the desirable range.

According to further embodiments of the present invention, if the original meal in the image/video taken by the image capture device 110 used to calculate the bolus dosage recommendation was not finished (i.e., the meal in the image/video was not entirely consumed), the user may take a second image of the unfinished meal and permit the bolus estimator 100 to calculate the difference between the original estimated carbohydrate intake, and the remaining carbohydrate value in the unfinished meal. The bolus estimator 100 may then recommend a course of action, if necessary, to counteract the potential excess insulin delivered based on the first estimate (e.g., recommend a sugar tablet dosage, an amount of juice to drink, a glucogon delivery dosage, etc.). According to additional embodiments of the present invention, a further image/video may be taken by the image capture device 110 if additional food item(s) (e.g., dessert) following the original meal is to be consumed by the user; the bolus estimator 100 may make a further bolus dosage recommendation for the additional food item(s) to be consumed based on the further image/video taken of that additional food item(s), taking into account the bolus dosage recommendation(s) already made.

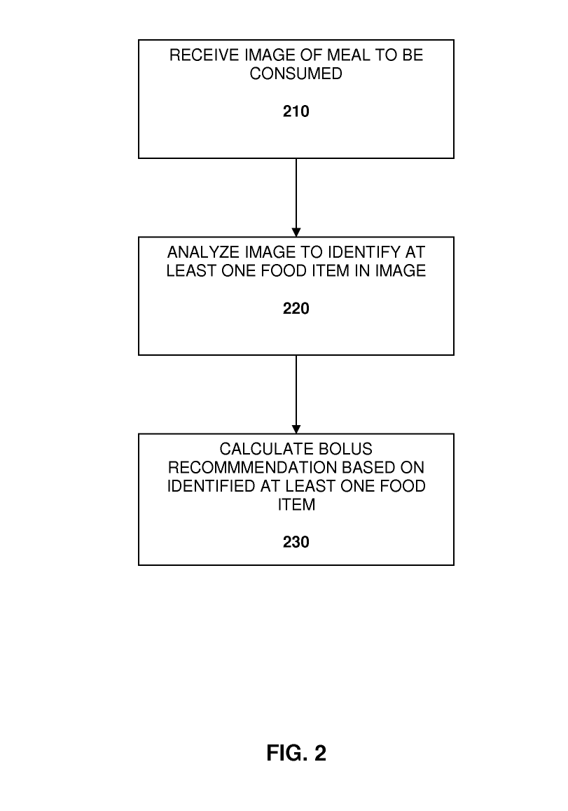

FIG. 2 illustrates a flow chart operation of a bolus estimator according to embodiments of the present invention. At step 210, an image/video of a meal to be consumed by a user is received by the bolus estimator 100. According to embodiments of the present invention, the bolus estimator 100 may include a digital camera, and the user may use the digital camera to take an image of a meal to be consumed. According to further embodiments of the present invention, the bolus estimator 100 with a digital camera may be integrated into a mobile phone combination device (e.g., “camera phone”), which also may include a blood glucose meter and/or an infusion pump programmer/controller. According to alternative embodiments of the present invention, the user may take an image of a meal to be consumed with a camera phone and then transmit the image (e.g., via Bluetooth, mini-USB cable, e-mail, picture messaging, etc.) to the bolus estimator 100. Any other suitable combinations with other devices and functionalities are also acceptable.

Once the image of the meal to be consumed is received by the bolus estimator 100, the image is analyzed using any suitable image recognition/image processing software to identify, at step 220, at least one food item in the image. The image may be analyzed locally at the bolus estimator 100, transmitted to another (local or remote) system for analysis, or a combination of both. According to embodiments of the present invention, the image may be transmitted from the bolus estimator 100 via any suitable wired or wireless transmission protocols (e.g., via Bluetooth, WiFi, satellite, cellular telephone, infrared, radio frequency, etc.) to a separate system for analysis. The analysis also may be partly performed locally at the bolus estimator 100 and partly performed on a separate system. For example, the bolus estimator 100 locally may store information (e.g., image of food, carbohydrate information, etc.) regarding the “Top 50” food items that the user is likely to consume, and if the bolus estimator 100 does not recognize a particular food item in an image, the image may be transmitted to a separate system for further analysis, where there may be a greater database capacity, and the results returned to the bolus estimator 100 via any suitable communication protocol.

Once the at least one food item is identified in the image, corresponding carbohydrate information regarding that at least one food item may be obtained, and at step 230, a bolus recommendation is calculated. Accordingly to various embodiments of the present invention, a portion size of the at least one food is determined and the carbohydrate value of the at least one food item based on its portion size may be obtained and used in the calculation of the bolus recommendation. If the bolus estimator 100 is unable to identify a meal to be consumed in the image/video, manual input of the meal information (e.g., carbohydrate value, protein value, fat value, caloric value, etc.) may be made by the user. The bolus recommendation may be transmitted to an insulin infusion device/pump or its programmer/controller, or alternatively, manually entered into an infusion device/pump or its programmer/controller. According to some embodiments of the present invention, the bolus estimator 100 may reside on the infusion device/pump itself, too, or any other suitable device, including consumer electronics and medical devices.

According to additional embodiments of the present invention, the bolus estimator 100 may be set in a “training” mode to aid a user in learning how to estimate carbohydrate values in food items and meals to be consumed. The bolus estimator 100 has access to images with known carbohydrate values, and these images may be presented to the user, e.g., on a display 130, and the bolus estimator 100 may prompt the user to estimate the carbohydrate value of the food items/meal shown in the image, and then inform the user how close the user's estimate was to the actual carbohydrate value. With practice, the user may learn to estimate carbohydrate values of food items and meals more accurately. Further description of systems and methods for calibrating bolus estimators/calculators may be found in U.S. patent application Ser. No. 12/343,904, filed Dec. 24, 2008, to Getschmann et al., entitled, “Systems and Methods for Providing Bolus Dosage Recommendations”, which is herein incorporated by reference in its entirety.

Images/videos taken by the image capture device 110 may be stored in a user's “food history” or “journal” (locally or remotely) according to embodiments of the present invention. An image/video of meals consumed by the user may be correlated to a timeline corresponding to the time when that meal was consumed by the user. If continuous glucose monitoring (CGM) data is available, this timeline of meal images/videos on a timeline also may be overlaid onto CGM data for that user so that the user's CGM data along with meal image/video information is available on the same timeline. For example, the user may scroll or select a point on the timeline indicating that a meal was consumed, and the image/video of the meal may appear when the user scrolls over or selects that point on the timeline. This information may be displayed on any suitable device, such as, for example, an infusion (insulin) pump, an infusion pump controller/programmer, a mobile phone, a PC, a PDA, a hospital monitor, etc.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. A method of providing bolus dosage recommendations for diabetics, comprising:
receiving an image of a meal to be consumed by a user;
analyzing the image to identify at least one food item in the image; and
calculating a bolus dosage recommendation based on the identified at least one food item in the image.

2. The method according to claim 1, wherein the image is captured by a digital camera.

3. The method according to claim 1, further including:
determining a portion size of the at least one food item in the image.

4. The method according to claim 3, wherein the portion size of the at least one food item in the image is determined by analyzing a reference object of a known size in the image.

5. The method according to claim 1, further including:
determining a carbohydrate value of the identified at least one food item in the image.

6. The method according to claim 5, wherein the carbohydrate value is obtained from a database.

7. The method according to claim 1, further including:
comparing the image with food images stored in a database to identify the at least one food item.

8. The method according to claim 7, wherein the food images stored in the database include corresponding known carbohydrate values.

9. The method according to claim 1, further including:
determining at least one of a protein value, a fat value, and a caloric value of the identified at least one food item in the image.

10. The method according to claim 1, wherein the method is implemented on a medical system.

11. The method according to claim 1, further including:
receiving a current blood glucose level of the user.

12. A bolus estimator, comprising:
an image capture device to capture an image of a meal to be consumed by a user;
a processor operatively coupled to the image capture device to analyze the image to identify at least one food item in the image, and to calculate a bolus dosage recommendation based on the identified at least one food item in the image; and
a display operatively coupled to the processor to display the bolus dosage recommendation.

13. The bolus estimator according to claim 12, wherein the processor further determines a portion size of the at least one food item in the image.

14. The bolus estimator according to claim 12, wherein the portion size of the at least one food item in the image is determined by analyzing a reference object of a known size in the image.

15. The bolus estimator according to claim 12, wherein the processor further determines a carbohydrate value of the identified at least one food item in the image.

16. The bolus estimator according to claim 15, wherein the carbohydrate value is obtained from a database.

17. The bolus estimator according to claim 12, wherein the processor further accesses a database to compare the image with food images stored in the database to identify the at least one food item.

18. The bolus estimator according to claim 17, wherein the food images stored in the database include corresponding known carbohydrate values.

19. The bolus estimator according to claim 12, wherein the processor further determines at least one of a protein value, a fat value, and a caloric value of the identified at least one food item in the image.

20. The bolus estimator according to claim 12, wherein the bolus estimator is integrated into a controller, an infusion pump, or a blood glucose meter.

21. The bolus estimator according to claim 12, wherein the bolus estimator is integrated into a mobile phone, a personal digital assistant (PDA), or a portable computer.

22. The bolus estimator according to claim 12, further including:
an input device to receive a current blood glucose level of the user.

23. The bolus estimator according to claim 12, wherein the image capture device is a digital camera.
